(19) United States
(12) Patent Application Publication
(10) Pub. No.: US 2009/0088703 A1
(43) Pub. Date: Apr. 2, 2009

(54) SKIN TREATMENT SYSTEM AND METHOD

(76) Inventor: KOUROS AZAR, Thousand Oaks, CA (US)

Correspondence Address: STETINA BRUNDA GARRED & BRUCKER, 75 ENTERPRISE, SUITE 250, ALISO VIEJO, CA, 92656, US

(21) Appl. No.: 12/209,105

(22) Filed: Sep. 11, 2008

Related U.S. Application Data
(60) Provisional application No. 60/993,667, filed on Sep. 13, 2007.

(51) Int. Cl. A61M 5/31

(52) U.S. Cl. 604/246

(57) ABSTRACT

A skin treatment system is provided for applying liquid medication through a controlled intra-dermal injection. The skin treatment system includes a microinjector unit with a proximal end and an opposing distal end. The proximal end of the microinjector unit may be press fitted or twist fit to a distal end of a tube via a luer lock mechanism. A fluid moving chamber is disposed within the microinjector unit. The fluid moving chamber is configured to receive liquid medication and distribute the liquid medication to a plurality of hypodermic needles. The plurality of hypodermic needles are operative for receiving the liquid medication from the fluid moving chamber and delivering the liquid medication to the skin of the patient. The plurality of hypodermic needles extends from the distal end of the microinjector unit. The distal end of the microinjector unit may encompass a variety of different shapes while the needles are maintained equidistantly spaced apart.

### CROSS-REFERENCE TO RELATED APPLICATIONS

The present application claims priority to Provisional Patent Application Ser. No. 60/993,667 entitled SKIN TREATMENT SYSTEM AND METHOD filed on Sep. 13, 2007.

### STATEMENT RE: FEDERALLY SPONSORED RESEARCH/DEVELOPMENT

Not Applicable

### BACKGROUND

The present invention relates generally to skin treatment through intra-dermal injections of liquid medication and, more particularly, to a method and system for performing intra-dermal injections of liquid medication using a microinjector unit to control and evenly apply medication, and especially botulinum toxin, to the skin.

A common form of hypodermic injection of medication is the intra-dermal injection. Various instruments, systems, and methods are well known in the art for providing intra-dermal injections. One such instrument includes a microinjector device which is a tool for infusion of very small amounts of fluids or drugs. Another instrument includes the well known small syringe. Intra-dermal injection using a small syringe attached to a short, fine gauge needle placed just below the skin surface is an extremely common medical procedure. Another type of device for administering liquid medication to a patient is the single use syringe design. Systems for delivering injections into humans have been in use for many years. The most commonly used system is a hypodermic needle attached to a small glass vial containing the liquid medication. To perform an injection, the needle is inserted into the tissue to the desired depth and the operator depresses a plunger inside the small glass vial containing the liquid medication to deliver the injection.

Intra-dermal injections are a well established region for depositing an injection for skin treatment. Intra-dermal injections place the solution or medication into the skin also known as the intra-dermal space. A needle and glass vial system can be effective for many types of intra-dermal injections because when the correct technique is employed, it can inject a predetermined amount of fluid (typical volumes range from 0.1 to 0.3 cc). Administering a proper intra-dermal injection using a conventional needle and glass vial injection system can be difficult. The space in which the tip of the needle must be placed is very small (about 1 mm). The shaft of the needle must be held at a very shallow angle with respect to the target surface. It is critical that the needle tip pass most of the way through the outer layer of skin, typically called the epidermis, but that the tip not penetrate completely through the dermis (the tissue layer that separates the skin layer from the underlying adipose layer or fat tissue), or the volume of solution to be injected will not be delivered entirely in the intra-dermal space. Thus, an intra-dermal injection with a needle and glass vial system requires an exacting technique from the user to give a proper injection. If the needle penetrates the dermis, the solution will enter the adipose layer (fat tissue). This happens frequently with conventional intra-dermal injections.

For some methods of skin treatment, it is important to limit the introduction of the solution or medication to the subcutaneous space. If intra-dermal medicine is allowed to diffuse to the subcutaneous space or to the underlying muscles, severe and debilitating side effects may be experienced by the patient. Thus, controlling the diffusion of intra-dermal medicine prevents side effects such as paralysis of the underlying muscles when undergoing different types of skin treatments. Besides the difficulty in regulating the diffusion of intra-dermal medicine, the skin treatment systems well known in the art require great skill to attempt to regulate extremely small injection doses through a single needle. Additionally, it is advantageous for the treatment of skin to deliver a total higher volume to the skin through a consistent dose of medication. Intra-dermal skin treatment can benefit from improved safety and effective distribution of medication such as Botox into the skin, as well as injection of dermal fillers of various viscosities and various depths such as sub-dermal and deep dermal. The injection of dermal fillers may improve facial contour, eliminating deep creases, wrinkles, rhytides, scars, depressions, or congenital deficiencies of the face by way of example.

Accordingly, there exists a need in the art for an improved method and system for performing intra-dermal injections of liquid medication using a microinjector device to control and evenly apply medication which addresses one or more of the above or related deficiencies.

### BRIEF SUMMARY

A skin treatment system is provided for applying liquid medication through a controlled intra-dermal injection. The skin treatment system includes a tube with a proximal end and an opposing distal end. The proximal end of the tube includes an opening for receiving a plunger that may be pushed or pulled to facilitate the injection of liquid medication into a patient. The skin treatment system also includes a fitting connector coupled to the distal end of the tube. The microinjector unit has a proximal end and an opposing distal end. The proximal end of the microinjector unit may be press fitted or twist fitted to the distal end of the tube with the fitting connector. The microinjector unit also includes a fluid moving chamber disposed therein. The fluid moving chamber is configured to receive liquid medication from the tube. The fluid moving chamber distributes the liquid medication to a plurality of hypodermic needles. The plurality of hypodermic needles are operative for receiving the liquid medication from the fluid moving chamber and delivering the liquid medication to the skin of the patient. The plurality of hypodermic needles extends from the distal end of the microinjector unit.

According to further embodiments, the plurality of hypodermic needles associated with the skin treatment system includes at least three hypodermic needles. The skin treatment system also defines a fluid path measured as the length between a point of entry for the liquid medication associated with the microinjector unit and a point of exit for the liquid medication located at a tip of the hypodermic needle attached to the distal end of the microinjector unit. Each hypodermic needle from the plurality of hypodermic needles has an inner diameter between 0.0635 mm and 0.1016 mm. The length of the hypodermic needles is between 0.95 mm and 1.2 mm. The limitation of the length for the hypodermic needles is intended to prevent diffusion of the liquid medication to the subcutaneous region or to underlying muscles where debilitating side effects may be experienced by skin treatment patients. The length of the needle improves the success of an intra-dermal injection. Another embodiment of the skin treatment system provides equidistance spacing for the plurality of hypodermic needles. In this regard, each hypodermic needle from the plurality of hypodermic needles is equidistantly spaced apart from each other.

In another embodiment of the skin treatment system, the fluid moving chamber distributes substantially the same quantity of fluid medication to each hypodermic needle. In other words, each hypodermic needle receives approximately the same amount of fluid volume of liquid medication from the fluid moving chamber. In certain novel applications, the medication comprises botulinum toxin that may be administered through the skin treatment system in a manner that is operative to improve skin texture, inhibit and/or eliminate sweating response and other aesthetic applications.

The skin treatment system and method may include a microinjector device comprising a plurality of shapes. By way of example the shapes may include but are not limited to an equilateral triangle, a star, a circle, a linear alignment or a curvilinear pattern. Irrespective of the shape, at least three equidistant hypodermic needles are included.

In another embodiment of the skin treatment system, the microinjector unit includes two guide notches at the distal end of the microinjector unit for lining up with previous injection points to ensure equal spatial distribution of the liquid medication.

The fitting connector of the skin treatment system may be a luer lock mechanism for press fitting or twist fitting the microinjector unit to the tube.

In another embodiment, the skin treatment system may be used to apply controlled intramuscular injection of liquid medication. The skin treatment system includes a microinjector unit having both a proximal end and an opposing distal end. Disposed within the microinjector unit is a fluid moving chamber configured to receive liquid medication. The fluid moving chamber is in mechanical communication with a plurality of hypodermic needles. The plurality of hypodermic needles receive the liquid medication from the fluid moving chamber. Each hypodermic needle associated with the microinjector unit has the same length. The length may range between 2 and 8 mm. The slightly longer length needles (2 to 8 mm) provide for actual intramuscular injection of medication such as botulinum toxin for the specific purpose of temporary paralysis in those muscles for therapeutic and aesthetic applications. The plurality of hypodermic needles extending from the distal end of the microinjector unit may be aligned in a linear, curvilinear or other shaped pattern with slightly longer needle lengths (2 to 8 mm) for the operative purpose of delivering more viscous dermal fillers such as hyaluronic acid gels into the subdermal space.

In another embodiment, a method for applying controlled intra-dermal liquid medication using a microinjector unit is provided. The microinjector unit includes a proximal end and an opposing distal end. A fluid moving chamber is disposed within the microinjector unit and configured to receive liquid medication for distribution to a plurality of hypodermic needles. The plurality of hypodermic needles is configured to extend from the distal end of the microinjector unit. The method begins by receiving the liquid medication at the proximal end of the microinjector unit. The method may continue with the distribution of the received liquid medication to the plurality of hypodermic needles via the fluid moving chamber. The method may conclude with the delivery of substantially equal fluid volume to equidistantly spaced portions of the skin of a patient from each hypodermic needle from the plurality of hypodermic needles.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is an explosed view of a skin treatment system;

FIG. 2 is a perspective view of the skin treatment system embodied in FIG. 1;

FIG. 3 is a cross-sectional view illustrating a microinjector unit;

FIG. 4 is a perspective view illustrating the microinjector unit with a pair of guide notches; and

FIG. 5 is a perspective view illustrating a star shaped microinjector unit.

### DETAILED DESCRIPTION

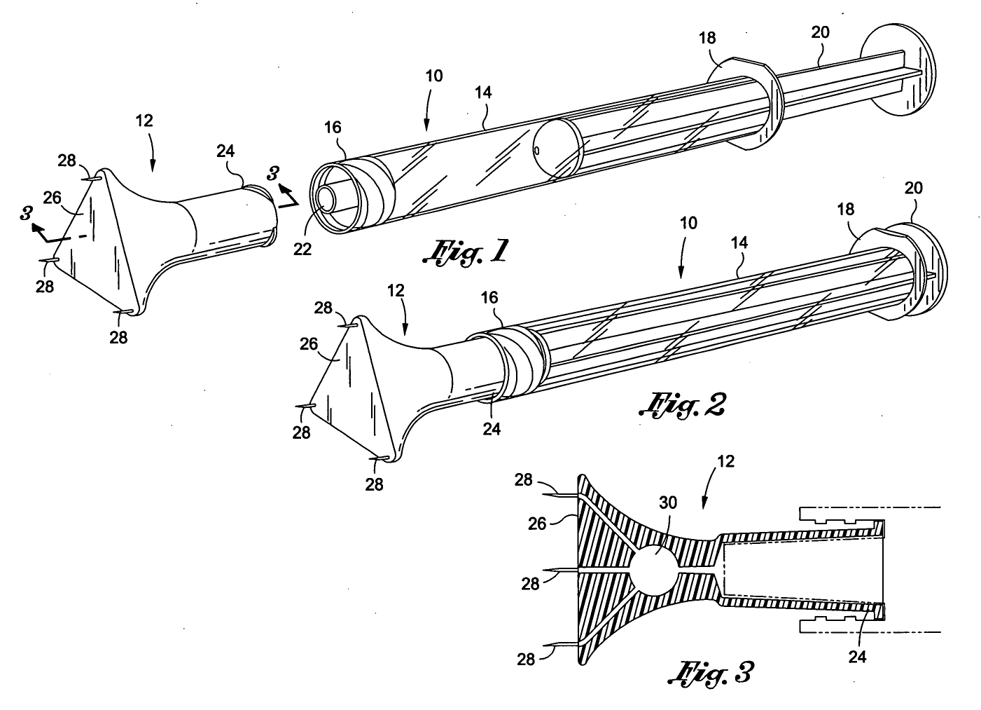

Referring now to the drawings wherein the showings are for purposes of illustrating embodiments of the skin treatment system and method only and not for purposes of limiting the same, shown in FIG. 1 is a skin treatment system 10. The skin treatment system 10 includes a microinjector unit 12. The microinjector unit 12 may be press fitted or twist fitted onto a tube 14. The tube 14 is typically formed from lightweight but durable material such as plastic and may be cylindrical in shape. The tube 14 may include a distal end 16 and an opposing proximal end 18. The proximal end 18 of the tube 14 includes an aperture or an opening for receiving a plunger 20. The plunger 20 may be pushed or pulled within the tube 14 for receiving liquid medication or delivering the liquid medication.

The distal end 16 of the tube 14 includes a fitting connector 22. The fitting connector 22 may be a luer lock mechanism. The microinjector unit 12 includes a proximal end 24 and an opposing distal end 26. The proximal end 24 of the microinjector unit 12 is configured to secure to the distal end 16 of the tube 14 via the fitting connector 22. As described above, the proximal end 24 of the microinjector unit 12 may be press fitted or twist fitted to the distal end 16 of the tube 14. FIG. 1 shows the microinjector unit 12 before being fitted to the tube 14 via the fitting connector 22. In FIG. 2, the microinjector unit 12 is secured to the tube 14.

The distal end 16 of the tube 14 may fit a standard luer lock design and is capable of attaching to a 1 cc or 3 cc syringe. The luer lock design provides a sealed lock between the microinjector unit 12 and the tube 14 which contains the liquid medication. The luer lock mechanism is designed to be leak proof.

The skin treatment system 10 including the microinjector unit 12 and the tube 14 may be designed for disposable and single use only. It may be packaged with a protective plastic shield to cover an array of hypodermic needles prior to use for blood borne pathogen precautions. In addition, various packaging elements may be incorporated to distinguish the various uses for the medication and to distinguish the benefits of the delivery system.

The distal end 26 of the microinjector unit 12 includes a plurality of hypodermic needles 28 for receiving and delivering the liquid medication to the skin of a patient. The plurality of hypodermic needles 28 on the distal end 26 or patient side of the microinjector unit 12 is designed to perform controlled intradermal injection of liquid medications. It is ideal (but not limited to) for injection of botulinum toxin or chemotherapy agents or acne medications. The lengths of the needles 28 are precisely specified to allow safe and controlled intradermal injection while limiting the introduction of the medication to the subcutaneous space. Furthermore, an embodiment of the skin treatment system uses short length needles 28 that are between 0.95 and 1.2 mm in length. The length of the needles 28 may limit the penetration of the needle to the dermis of the skin and limit exposure of the subcutaneous space and musculature to the injected medication. This limit corresponding to the injection depth enhances the safety of the skin treatment system 10 and expands its use to non-experienced health care providers such as physicians' assistants, medical assistants and nurses.

For the purposes of injecting intradermal botulinum toxin, the microinjector unit 12 controls the diffusion of intradermal medicine and minimizes possible side effects such as paralyzing the underlying muscles. Variations in the size and length of the needles may be required to adjust for viscosity of the fluid being injected and the required injection force and timing. The goals and depth of penetration of the injectable material may vary from that described for the applications to botulinum toxin.

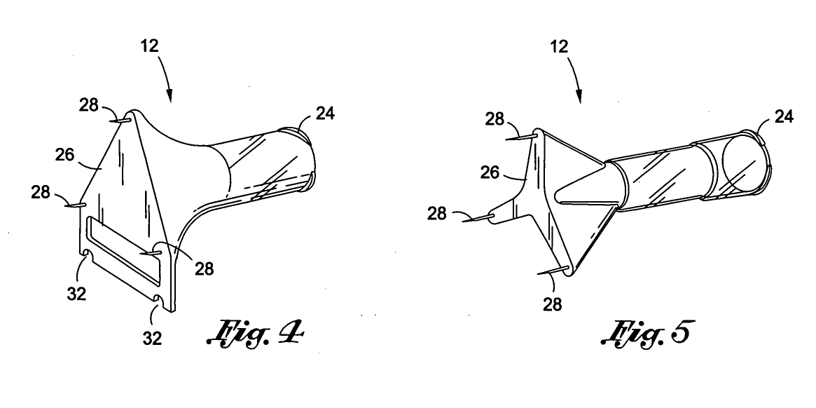

Referring now FIG. 3, a cross-sectional view illustrating a fluid moving chamber 30 disposed within the microinjector unit 12 is provided. The fluid moving chamber 30 is contained within the microinjector unit 12 and is connected to the plurality of hypodermic needles 28. Within the inner workings of the microinjector unit 12, the fluid moving chamber 30 readily allows equal distribution of fluid volume to each hypodermic needle from the plurality of hypodermic needles 28 during the injection process. By delivering a total higher volume to the skin through this skin treatment system 10, the person performing the injection will deliver a more consistent dose to the skin as larger volumes are more easily measured in practical day to day use. This would be in contrast to attempting to regulate extremely small injection doses through a single needle. The fluid moving chamber 30 may allow variations in fluid resistance either through decreasing the caliber of the fluid path in diameter or by increasing the total length of the fluid path from the entry of the microinjector unit 12 to the exit at the needle tip. This could be accomplished by a spiral or tortuous fluid path to increase the total length of the fluid path within the microinjector unit 12 without compromising the actual size of the microinjector unit 12.

Referring now to FIGS. 2, 4 and 5, the distal end 26 of the microinjector unit 12 may be comprised of various shapes including but not limited to an equilateral triangle, a circle, a star shape, a linear pattern, square, square array or any other geometric pattern with short hypodermic needles 28 extending from the distal end 26. The geometry of the design allows predictable calculation of the diffusion properties of the medication. The microinjector unit 12 can be used in a variety of geometries to inject dermal fillers of various viscosities and various depths such as sub-dermal and deep dermal. The distance between the plurality of hypodermic needles 28 may be kept equidistant. If the plurality of hypodermic needles 28 is equidistantly spaced the mathematics is simplified and the injectable dose can be readily calculated based on the geometry of the microinjector unit 12.

The shape of the microinjector unit 12 can be tailored to the specific application, for example: for intradermal injection of medications on the patient's face, the equilateral triangle or star shape design will allow the face to be divided into aesthetic subunits thereby treating the entirety of the face without missing small areas such as the peri-nasal or glabellar regions. The star shape design minimizes the plastic material around the hypodermic needles 28 and allows entry into corners of the face without inhibition by excess plastic between the injection needles 28. A variation of the microinjector unit 12 contains an additional plastic template with two guide notches 32 that may be lined up with two previous injection points to insure equal spatial distribution of the medication. In this variation of the microinjector unit 12, the distance from the two plastic notches to the hypodermic needles 28 on the distal end 26 is equal to the length of one side of the equilateral triangle portion of the microinjector unit 12.

The microinjector unit 12 is designed for simultaneously delivering equal amounts of medication to multiple points inside the human body using a tube 14 that is secured to the microinjector unit 12 with a fluid moving chamber 30. The fluid moving chamber 30 acts as a reservoir attached to at least three needles which carry the fluid to their destination. The design is simple thereby eliminating the need for valves and complicated geometries in order to minimize manufacturing costs and enhance marketability. The design allows for the flow rate of medication to all injection points to remain constant regardless of exit conditions. The microinjector unit 12 provides a large enough pressure drop across the entry and exit points, the pressure differential between the exit pressure and the various injection/delivery points is negligible. The pressure drop is achieved by lengthening the delivery ducts, which in one embodiment is a 32 gauge hypodermic needle. For such a needle, the inner diameter can vary from 0.0635 mm to 0.1016 mm.

The hypodermic needle forms a smooth circular pipe. In a simulation testing the properties of the skin treatment system 10, the fluid (medication) was assumed to have the physical properties of water at room temperature. In the simulation the desired amount of medication to be delivered via a single hypodermic needle was 0.05 cc at a rate of one injection in 2-4 seconds. Because the needle's inner diameter was predetermined by the choice of the 32 gauge hypodermic needle, the corresponding Reynolds number (the ratio of inertial forces to viscous forces) was calculated to be approximately 300. This implies the flow was fully laminar. In addition, transient times were assumed to be insignificant such that only the fully developed solution was considered.

For fully developed laminar flow in a round needle, the Navier-Stokes equations gives us the following velocity distribution in equation (1):

$${v}_{z} = \frac{1}{4 μ} ( \frac{d P}{d z} ) ( {r}^{2} - {( D / 2 )}^{2} ) .$$

Where v_{z} is flow velocity along the needle, μ is viscosity, dP/dz is the pressure gradient along the needle, r is the radial position, and D is the diameter of the needle.

This can be related to the overall volumetric flow rate in the needle through integration in equation (2):

$$Q = 2 π {\int }_{0}^{D / 2} {v}_{z} r d r = \frac{- π {D}^{4}}{128 μ} ( \frac{d P}{d z} ) .$$

Q is the volumetric flow rate. If the pressure gradient is assumed to be linear then equation (3):

$$\frac{d P}{d z} = \frac{Δ P}{L} ,$$

represents the pressure drop across the entire needle and L is the length of the needle, ΔP is the change in pressure. Combining equations 2 & 3 and solving for pressure drop, the following equation (4) is attained:

$$Δ P = \frac{- 128 μ QL}{π {D}^{4}} .$$

If there are ‘i’ number of needles, the total flow rate, Qₜₒₜ, is given by equation (5): Qₜₒₜ=Q₁+Q₂+ . . . +Qᵢ. Additionally, the pressure in the reservoir, P₀ must satisfy equation (6): P₀=P₁−ΔP₁=P₂−ΔP₂= . . . =Pᵢ−ΔPᵢ where Pᵢ is the exit pressure for needle i and ΔPᵢ is the corresponding pressure drop. The exit pressure the needle might experience was given by the pressure inside small blood vessels which is around 20 mm hg³. Using equations 4-6 and a given Qₜₒₜ, the individual flow rates for each needle were solved for. The total flow rate can be calculated by equation (7):

$${Q}_{tot} = \frac{\times i}{t}$$

where [symbol] is the volume of medication desired for a single injection and t is the total time the injection should take. The force required to actuate the syringe to achieve the desired flow rate can be determined by F=πR²P₀ where R is the radius of the syringe. The radius of the syringe was assumed to be 1.5 cm for all the calculations. The force should be kept well below the average human body's maximum grip strength of 250 lbs.

A Matlab code was written for the above simulation/experiment to automatically solve for the flow rates in each needle. The code allowed for the number of needles to be varied as well as all the geometries and pressures. A Gaussian distribution was used for random assignment of the exit pressures about the expected mean as well as prescribed pressures for investigation of a particular scenario. A similar distribution was used for the variability of inner needle diameter; however, all needles for a given calculation where assigned the same diameter since the needles will most likely have the same length of tubing. This allowed for the needle tolerances to be included without an overestimation as to their significance.

Graph 1 below shows the envelope of possibility for delivered medication plotted versus the needle lengths for a 3 needle configuration. Beyond needle lengths of 1 cm, the envelope is within ±10% of the mean 0.05 cc. Graph 2 below shows envelope of possibility for the force required to sustain the desired flow rate versus the needle lengths. For needle lengths up to 10 cm, the required force is well below the maximum attainable by the average human. Graph 1 represents medication delivery envelope versus tube length. The simulation used three 32 gauge needles with an injection time of 4 seconds.

Graph 3 above represents medication delivered for two worst case scenarios. Each line represents the amount of fluid delivered per needle. Lines representing two needles are half the total output of both needles combined. In a worst case scenario, two needles will pierce the skin while one needle does not pierce the skin. Another worst case scenario occurs when only one of the three needles pierces the skin while the other two needles do not pierce the skin. This confirms that beyond 1 cm, the amount of fluid leaving the needles will be within a ±10% range of the desired mean. For further improvement in tolerance, longer needles may be used. An increase in the number of needles does not affect the envelopes of possibility for medication delivery, see Graph 4 below. It has a similar effect on the required force. As long as the force on the plunger 20 can be kept constant, the flow rates for every needle will be constant and within the tolerance determined by the needle lengths. The Graph 4 below represents medication delivery envelope versus tube length. Again, the simulation used thirty 32 gauge needles with an injection time of 4 seconds. This and Graph 1 appear to show no discrepancies.

From these simulations it can be concluded that a simple device which delivers equal amounts of medication to multiple points inside the human body is feasible. In order for the device to be within ±10% range of the desired medication, the needles which carry the medication from the reservoir are recommended to have a diameter corresponding to 32 gauge and a length of at least 1 cm long. An increase in the length will result in a smaller margin of error. Up to lengths of 10 cm, the human body should still be able to work a 3 cm syringe to deliver the medication. The minimum number of needles analyzed was three. Any number of needles beyond that should still exhibit the same behavior as long as the required force and flow rates are achieved.

In further experiments, three different needle lengths were tested. Three prototypes were created each with different needle lengths (1 cm, 2 cm, and 3 cm). All needles were made from 32 gauge stainless steel tubes. They were cut and filed using a high speed dremel and then attached to a plastic lure with epoxy. A test rig was also created with a reservoir which could be raised to the pressure of human blood vessels. The reservoir is attached to rubber tubing which the prototypes could penetrate for testing. During a test, the tube 14 is filled with purified water and a prototype needle tip attached on one of the three needles is allowed to penetrate the rubber tubing while the other two needles are allowed to sit in a cup exposed to atmospheric pressure. The plunger 20 is then pressed at a constant rate until most of the fluid is drained out. Afterwards, the cup and the rubber tubing are weighed separately to calculate the amount of fluid delivered. The following graph shows the data from the tests as well as the simulated performance for similar flow conditions. The Graph 5 below represents the relationship between the tube length and mean delivered as a percentage.

In another embodiment, the microinjector unit 12 is used primarily for botulinum toxin injection for treating the skin. It is contemplated that the microinjector 12 unit is designed to inject any liquid medication that requires even distribution to areas of skin intra-dermally. However, the microinjector unit 12 has some unique elements that are designed specifically to enhance and expand the applications and safety of botulinum toxin for facial aesthetics. Furthermore, the skin treatment system 10 can be used to increase the safety margin on intradermal botulinum toxin injection for treatment of hyperhydrosis. This is particularly useful in the palms of the hand where overdose or aberrant distribution of the drug can have significant side effects on the muscles of the hand such as the thenar muscles.

With regard to the use of the skin treatment system 10 for intradermal injection of botulinum toxin and its aesthetic applications, the system may provide improved skin texture through the inhibition of both sweat gland function and pilomotor responses such as piloerection. To establish this, the mechanism of action of botox on the elements of the skin and the thickness of the skin must be reviewed in detail below.

It is known that intradermal injection of botulinum toxin will inhibit and/or eliminate the sweating response in a dose dependent manner. Both the sweating response and pilomotor responses are controlled by efferent sympathetic nerves in which the terminals release a neurotransmitter called acetylcholine as described in Neural control and mechanisms of eccrine sweating during heat stress and exercise by Shibasaki, M., Wilson, T. E., Crandall, C. G. in the Journal of Applied Physiology 100: 1692, 2006, and Structure and functions of the cutaneous nervous system by Reznik, M. Pathol Biol (Paris), 44 (10): 831, 1996, the teachings of which are expressly incorporated herein by reference. Piloerection which causes the hair to stand up is usually seen in response to cold, increased sympathetic tone, acute fear or narcotic withdrawal and is also controlled by sympathetic nerve terminals that secrete acetylcholine. These nerve terminals cluster around secretory coils of the sweat gland, ducts and the arrector pili muscles. As the acetylcholine arrives at the post-synaptic junction, it binds to muscarinic acetylcholine receptors and activates the eccrine sweat gland and arrector pili muscle. The pre-synaptic release of acetylcholine is very effectively blocked by botulinum toxin thus reversibly shutting down the sweat gland and pilomotor responses. Once the sweat gland response and pilomotor response has been shut down, the pore which is part and parcel of the sweat duct will shrink. Over a period of several months, the arrector pili muscle will atrophy and contribute to the shrinking of the perceived pore size. It is well known that irregular skin texture correlates with the presence of enlarged pores as described in Relationships between visual and tactile features and biophysical parameters in human facial skin by Ambroisine, L., Ezzedine, K., Elfakir, A., Skin Research and Technology, 13: 176, 2007, expressly incorporated herein by reference. Thus, the botulinum toxin induced shrinking of the pores and the atrophy of the arrector pili muscle will lead to a smoother skin texture. This improvement in skin texture has been clinically observed in large numbers of patients on the forehead in patients who regularly undergo botox injection of the frontalis muscle for horizontal forehead rhytids. This is further supported by the fact that the forehead has the highest density of eccrine sweat glands of any other part of the face and one of the highest on the body. In addition, the summated contraction of the pore size over the volume of the face may also lead to a perceived tightening of minute areas of skin laxity. This overall improvement in skin texture and contour has a very desirable effect on the aesthetic appearance of the face.

In order to effect these changes on the skin, the botulinum toxin must be injected intradermally while minimizing the diffusion to underlying muscles of the face and hand. Therefore, the microinjector unit 12 is needed to limit the depth of injection and the spatial diffusion over the surface area of the face. In order to establish an understanding of the appropriate needle length 28 for the distal end 26, a review of studies analyzing human skin thickness is discussed below.

Many studies have been performed analyzing human skin thickness using histologic studies, high resolution ultrasound, confocal imaging and cadaver studies. These studies have also addressed differences in thickness between different regions of the body, different regions within the face itself and differences in thickness between patients of differing ages and ethnicities. On cadaver studies, average skin thicknesses on clinically relevant areas of the face ranged between 0.73 mm and 1.22 mm with an average thickness of 0.97 mm as described in Analysis of Facial Skin Thickness: Defining the Relative Thickness Index by Ha, R. Y., Nojima, M. D., K., Adams, Jr., W. P., Plastic and Reconstructive Surg., 115(6): 1769, 2005, expressly incorporated by reference. In this study the cheeks had a measured thickness of 1.07 mm, upper lip 0.83 mm and chin 1.15 mm. On other parts of the body such as the forearm, the mean dermal thickness was 0.92 mm at a mean age of 60 with a standard deviation of 0.18 as described in Can dermal thickness measured by ultrasound biomicroscopy assist in determining osteoporosis risk? by Cagle, P. E., Dyson, M., Gajewski, B., Skin Research and Tech., 13: 95. 2007, expressly incorporated herein by reference. By contrast, hip thickness was notably thicker at 1.69 mm on average based on high frequency in vivo ultrasound described in Scarring occurs at critical depth of skin injury: Precise measurement in a graduated dermal scratch in human volunteers by Dunkin, C. S., Pleat J. M., Gillespie, P. H., Journal of the American Society of Plastic and Reconstructive Surgery, 119 (6): 1722, 2007, expressly incorporated herein by reference. The validity of high frequency ultrasound as an accurate measure of skin thickness has been proven by comparison to histologic sections for accuracy. [Overgaard Olsen, 1995 and Milner et al. 1997]. Furthermore, the thickness of the epidermis has been shown to vary only minimally by a factor of only 10 micrometers on average as described in In vivo data of epidermal thickness evaluated by optical coherence tomography: Effects of age, gender, skin type, and anatomic site by Gambichler, T., Matip, R., Moussa, G., Journal of Dermatological Science, 44 (3): 145, 2006, expressly incorporated herein by reference. This suggests that variations in skin thickness by age are primarily due to changes in dermal thickness. High frequency ultrasound studies of skin thickness show no statistical significance between races or ethnicities as described in In vivo biophysical characterization of skin physiological differences in races by Berardesca, E., De Rigal, J., Leveque, J. L., Dermatologica, 182 (2): 89, 1991, expressly incorporated herein by reference. Thus, based on these studies and an extensive review of the literature with regard to skin thickness, it is found to be a reasonable estimate that intra-dermal injection of a depth of 0.95 to 1 mm will result in a margin of error to insure appropriate intra-dermal injection in most clinically relevant parts of the face.

When botulinum toxin is injected intradermally, it will diffuse to the surrounding dermis and some small quantity may diffuse into the subcutaneous space. The effect of this diffusion is dependent on the volume of injection, the concentration of the solution and the size of the subcutaneous space. Using the microinjector unit 12, a predictable equation can be formulated based on the equilateral triangle geometry to predict the diffusion of the solution. The dose to be injected is safe and effective in the range of 0.5-0.8 mU/cm². This is based on studies performed for the treatment of hyperhydrosis of the palm as described in Side-effects of intradermal injections of botulinum A toxin in the treatment of palmar hyperhidrosis: a neurophysiological study by Swartling, C., Farnstrand, C., Abt, G., European Journal of Neurology, 8: 451, 2001, expressly incorporated herein by reference. In this study, careful EMG studies were performed to measure weakness of thenar muscles of the hand after intra-dermal injection of botulinum toxin. There were no observed side effects below 0.5 mU/cm². Furthermore, recent clinical studies have shown no undesirable effects of botulinum toxin when injected at higher volumes of dilution as described in A randomized, evaluator-blinded, two-center study of the safety and effect of volume on the diffusion and efficacy of botulinum toxin A in the treatment of lateral orbital rhytides by Carruthers, M. D., A., Bogle, M. D., M, Carruthers, J. D., Dermatologic Surgery, 33: 567, 2007, expressly incorporated herein by reference. Thus the volume may be adjusted to give the optimal accuracy for the health care provider performing the injection.

While an illustrative embodiment of the invention has been described in detail herein, it is to be understood that the inventive concepts may be otherwise variously embodied and employed.

What is claimed is:

1. A skin treatment system for applying controlled intra-dermal liquid medication comprising:
a tube having a proximal end and an opposing distal end, the proximal end having an aperture for receiving a plunger;
a fitting connector coupled to the distal end of the tube; and
a microinjector unit having a proximal end and an opposing distal end, the proximal end of the microinjector unit being coupled to the distal end of the tube via the fitting connector, the microinjector unit having a fluid moving chamber disposed within, the fluid moving chamber configured to receive liquid medication from the tube and distribute the liquid medication to a plurality of hypodermic needles being operative for receiving and delivering liquid medication to the skin of a patient, the plurality of hypodermic needles extending from the distal end of the microinjector unit.

2. The skin treatment system according to claim 1, wherein the plurality of hypodermic needles includes at least three hypodermic needles having substantially equal dimensions.

3. The skin treatment system according to claim 1, further comprising a fluid path defined by length between a point of entry for the liquid medication into the microinjector unit and a point of exit for the liquid medication located at a tip of the hypodermic needle attached to the distal end of the microinjector unit.

4. The skin treatment system according to claim 1, wherein each hypodermic needle from the plurality of hypodermic needles has an inner diameter between 0.0635 mm and 0.1016 mm.

5. The skin treatment system according to claim 1, wherein the length of each hypodermic needle from the plurality of hypodermic needles is between 0.95 mm and 1.2 mm.

6. The skin treatment system according to claim 1, wherein the plurality of hypodermic needles are spaced equidistant from each other.

7. The skin treatment system according to claim 1, wherein the fluid moving chamber distributes substantially equal fluid volume of liquid medication to each needle from the plurality of hypodermic needles.

8. The skin treatment system according to claim 1, wherein the distal end of the microinjector unit has two guide notches for lining up with previous injection points to ensure equal spatial distribution of the liquid medication.

9. The skin treatment system according to claim 1, wherein the fitting connector is a luer lock mechanism.

10. The skin treatment system according to claim 1, wherein the tube is cylindrical in shape.

11. The skin treatment system according to claim 1, wherein the distal end of the microinjector unit is shaped as an equilateral triangle.

12. The skin treatment system according to claim 1, wherein the distal end of the microinjector unit is shaped as a circle.

13. The skin treatment system according to claim 1, wherein the distal end of the end of the microinjector unit is shaped as a star.

14. The skin treatment system according to claim 1, wherein the plurality of hypodermic needles are aligned linearly.

15. The skin treatment system according to claim 1, wherein the plurality of hypodermic needles are aligned in a curvilinear pattern.

16. A skin treatment system for applying controlled intramuscular injection of liquid medication comprising:
a tube having a proximal end and an opposing distal end, the proximal end having an aperture for receiving a plunger;
a fitting connector coupled to the distal end of the tube; and
an microinjector unit having a proximal end and an opposing distal end, the proximal end of the microinjector unit being coupled to the distal end of the tube via the fitting connector, the end piece having a fluid moving chamber disposed within, the fluid moving chamber configured to receive liquid medication from the tube and distribute the liquid medication to a plurality of equidistantly spaced hypodermic needles, each hypodermic needle having the same length between 2 and 8 millimeters, the plurality of hypodermic needles extending from the distal end of the microinjector unit.

17. A method for applying controlled intra-dermal liquid medication using a microinjector unit having a proximal end and an opposing distal end, the microinjector unit having a fluid moving chamber disposed within, the fluid moving chamber configured to receive liquid medication and distribute the liquid medication to a plurality of hypodermic needles, the plurality of hypodermic needles extending from the distal end of the microinjector unit, comprising:
receiving liquid medication at the proximal end of the microinjector unit;
distributing the received liquid medication to the plurality of hypodermic needles via the fluid moving chamber; and
delivering substantially equal fluid volume to equidistantly spaced portions of the skin of a patient from each hypodermic needle from the plurality of hypodermic needles.
